(19) United States
(12) Patent Application Publication
(10) Pub. No.: US 2018/0192873 A1
(43) Pub. Date: Jul. 12, 2018

(54) MONITORING PHYSIOLOGOGY

(71) Applicant: Cambridge Temperature Concepts Ltd, Cambridge, Cambridgeshire (GB)

(72) Inventors: Oriane Elisabeth CHAUSIAUX, Cambridge (GB); Matthew Neil SARKAR, Cambridge (GB); Shamus Louis Godfrey HUSHEER, London (GB)

(21) Appl. No.: 15/520,683

(22) PCT Filed: Oct. 23, 2015
(86) PCT No.: PCT/GB15/53187

§ 371 Date: Apr. 20, 2017

(30) Foreign Application Priority Data
Oct. 24, 2014 (GB) 1419008.6

(51) Int. Cl. A61B 5/00; A61B 5/01; A61B 5/021

(52) U.S. Cl. CPC A61B 5/002; A61B 2503/20; A61B 5/7275; A61B 5/4815; A61B 5/7289; A61B 5/7282; A61B 5/746; A61B 5/0008; A61B 5/4842; A61B 5/6832; A61B 5/6831; A61B 5/02125; A61B 2560/0252; A61B 2562/0271; A61B 2562/0219; A61B 2560/0475; A61B 5/01

(57) ABSTRACT

A physiological monitoring system comprising a sensor device for attachment to the skin of a subject, the sensor device comprising at least a first sensor for sensing a physiological parameter of the subject and a processing device configured for analysing data sensed by the sensor by means of a predetermined algorithm to estimate whether the data is indicative of a disease condition, and on detecting such a condition to generate an alert. It is further described a method for monitoring a plurality of subjects comprising the step of providing each subject with a sensor device as referred to above and then analysing data sensed by the sensors such as to preferentially generate an alert if the data received from multiple ones of the sensors demonstrate a contemporaneous trend indicative of a disease condition.

This invention relates to monitoring physiological functions.

It is known to monitor physiological functions of a subject's body in order to assess the health or capabilities of the subject. Physical functions that are commonly monitored include body temperature, activity, respiration rate, heart rate, perspiration state, blood pressure, blood oxygen content and hydration level. Changes in some of these parameters are known to be suggestive of certain disease conditions. For example, it is known that a subject who is suffering from an infection may experience a rise in body temperature.

When a patient is suffering from a particularly serious infectious disease, the patient may be isolated to help prevent transmission of the disease to healthcare workers. The patient may be placed in an isolation tent, sealed from the surrounding environment, which allows infectious material passed from the patient to be tightly controlled. Meanwhile, it may be prudent to monitor the health of workers who are treating the patient in order to check that the disease has not been transmitted to them.

These precautions raise a number of practical problems. First, when a patient is under strict isolation it is difficult to obtain measurements of their condition. For example to take the patient's temperature doctors might use a remote reflectivity-based infra-red thermal detector that is shone onto the patient's skin from outside the isolation cordon. This approach is relatively inaccurate. Second, it is difficult to reliably monitor the health of the workers. For example, each worker might be asked to take their own temperature once a day and to raise an alert if they identify an increase in temperature. However, (i) it is difficult to know that the workers are adhering to this regime; (ii) the inaccuracy of normal body temperature measurements means that the infection might be in an advanced stage before the worker detects it, whereas it would be desirable for infection to be detected at the earliest possible moment; and (iii) the point at which a worker might self-report potential infection depends on the attitude of the worker: some workers might be hesitant to report that they might be infected, whereas others might be overly cautious. Furthermore, there are technical difficulties in accurately measuring the physiological functions of patients in isolation or workers who are treating them. If the patient is in a sealed environment of small volume then the temperature and humidity of that environment may vary significantly depending on the state of the patient themselves. That can affect the measurements of the patient. Similarly, the workers will frequently wear enclosed barrier suits which can become hot and humid over time, affecting measurements of the workers.

There is a need for an improved way to monitor the physiological functions of subjects such as patients and healthcare workers.

According to one aspect of the present invention there is provided a physiological monitoring system comprising: a sensor device for attachment to the skin of a subject, the sensor device comprising at least a first sensor for sensing a physiological parameter of the subject; and a processing device configured for analysing data sensed by the sensor by means of a predetermined algorithm to estimate whether the data is indicative of a disease condition, and on detecting such a condition to generate an alert.

The sensor device may comprise a body and a second sensor, the second sensor being for sensing the same physical quantity as the first sensor, the first sensor being located adjacent to a first surface of the body and the second sensor being located adjacent to an opposing surface of the body.

The sensor device may comprise an accelerometer.

The system may be configured to selectively analyse data gathered by the sensors at times dependent on data gathered by the accelerometer.

The system may be configured to, in dependence on data sensed by the accelerometer during a sleep session of the subject, form an estimate of the quality of the subject's sleep during that session and to estimate whether the data is indicative of a disease condition in dependence on that sleep quality estimate.

The sensors may be temperature sensors.

The system may be configured to selectively analyse temperature data gathered at times of relatively low acceleration as sensed by the accelerometer.

The system may be configured to form an estimate of the lowest temperature attained by a wearer of the device during a sleep session. The estimation of whether the data is indicative of a disease condition may be dependent on that temperature estimate.

The system may be configured to form an estimate of the basal body temperature of a wearer of the device during a sleep session. The estimation of whether the data is indicative of a disease condition may be dependent on that temperature estimate.

The system may be configured to store data previously sensed by the sensor device. The estimation of whether the data is indicative of a disease condition may be dependent on detecting a deviation of a predetermined form between data currently sensed by the sensor device and that previously sensed data. The deviation of a predetermined form may be a deviation by greater than a threshold from an average formed over at least part of the previously sensed data.

The system may be configured to store data previously sensed in respect of multiple subjects and determined to be indicative of the onset of a disease condition. The estimation of whether the data is indicative of a disease condition may be dependent on detecting a commonality of a predetermined form between data currently sensed by the sensor device and that previously sensed data.

The system may be configured to generate an alert indicating the disease condition with the data for which a commonality has been detected in data currently sensed by the sensor.

The system may be configured to form modified temperature data by modifying data sensed by the first sensor in dependence on contemporaneous data sensed by the second sensor. The estimation of whether the data is indicative of a disease condition may be dependent on the modified temperature data.

The modified temperature data may be an estimate of heat flow from the subject to the environment.

The system may be configured to analyse data received from the sensor to compare the received data with one or more data patterns whose definitions are stored as being indicative of a device that is being worn by a human subject, and to generate an alert on detecting a deviation of a predetermined form between data currently sensed by the sensor device and one or more of those patterns.

The system may comprise a second sensor device for attachment to the skin of a subject. The second sensor device may comprise at least a third sensor for sensing a physiological parameter of the subject. The first and second sensor devices may be adapted to be attached to the body of the subject at different locations. The system may be configured to analyse data received from the sensors in dependence on their locations on the body of the subject to estimate whether the data is indicative of a disease condition.

The disease condition may be the onset of initial symptoms of a disease. The disease may be a viral infection. Alternatively the disease condition may be an exacerbation of a chronic disease.

According to a second aspect of the present invention there is provided a method for monitoring a plurality of subjects, comprising: providing each subject with a sensor device for attachment to the skin of the subject, the sensor device comprising at lease a first sensor for sensing a physiological parameter of the respective subject; and analysing data sensed by the sensors by means of a predetermined algorithm to estimate whether the data from each sensor is indicative of a disease condition, and on detecting such a condition to generate an alert, the analysis being such as to preferentially generate an alert if the data received from multiple ones of the sensors demonstrate a contemporaneous trend indicative of a disease condition.

The present invention will now be described by way of example with reference to the accompanying drawings. In the drawings:

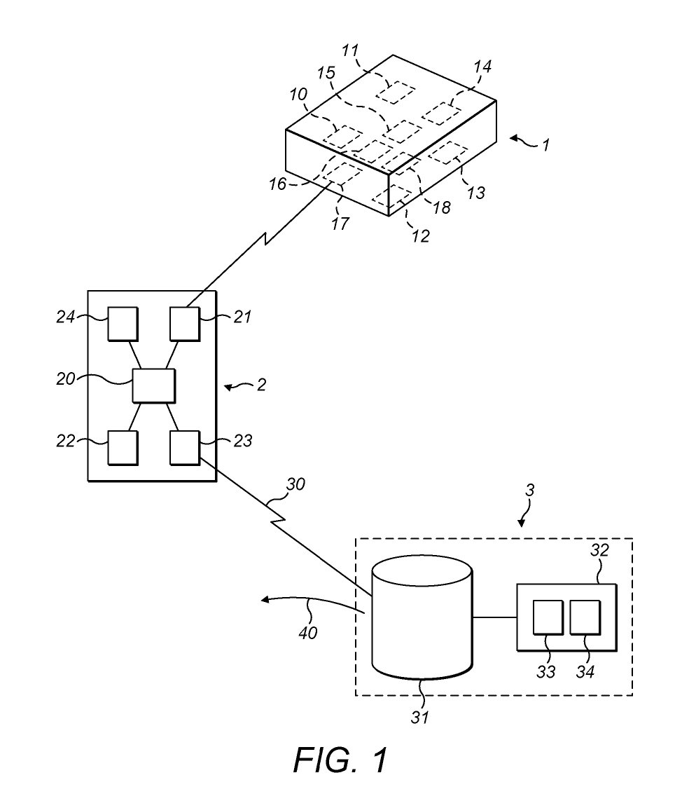

FIG. 1 shows a system for monitoring a subject's physiology.

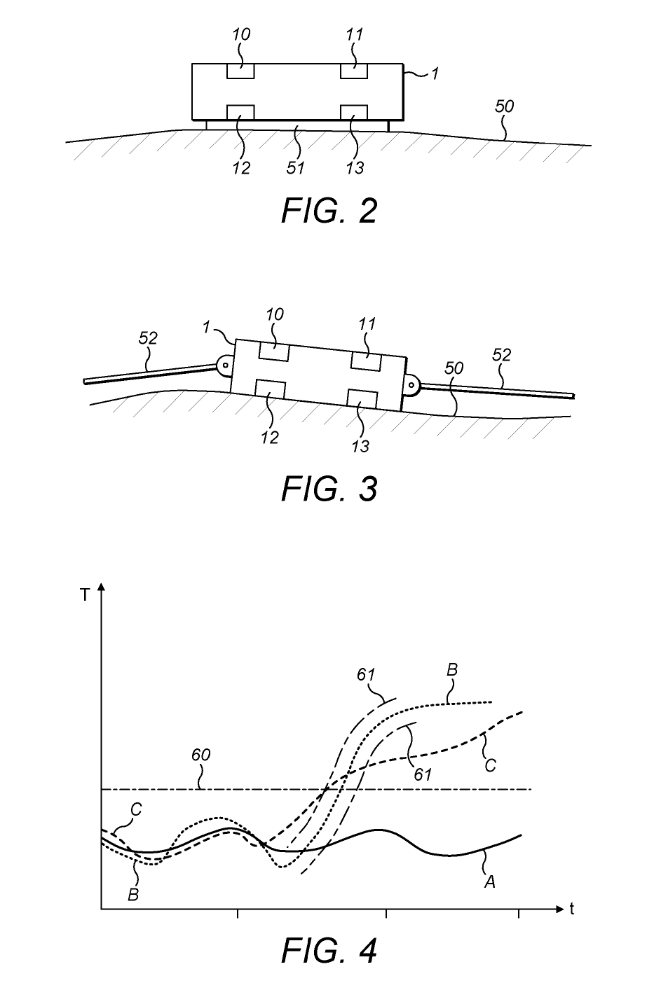

FIGS. 2 and 3 show ways of attaching a datalogger to a subject.

FIG. 4 illustrates temperature profiles for a number of subjects.

FIG. 1 is a schematic diagram of a system for monitoring the physiology of a subject. The system comprises a portable sensing device 1, a relay device 2 and a monitoring data centre 3.

The portable sensing device 1 is configured to be carried by a subject and comprises a number of sensors 10, 11, 12, 13, a battery 14, a processor 15, a memory 16 and a wireless transceiver 17. The battery provides an energy source for the other components so that the portable sensing device can be self-contained. The processor 15 is configured to execute software stored in a non-transient form in memory 16 in order to cause the device to perform its functions. The processor communicates with the sensors 10-13 to receive data gathered by the sensors and with the wireless transceiver to permit the processor to wirelessly transmit and receive data. The transceiver could operate in the ISM (industrial, scientific and medical band) and could be a Bluetooth (e.g. Bluetooth Low Energy) or IEEE 802.11 transceiver. It could operate according to other protocols.

The relay device serves to relay communications from the sensing device to the monitoring data centre 3. The relay device comprises a processor 20, a wireless transceiver 21, a memory 22 and an uplink transceiver 23. The processor 20 is configured to execute software stored in a non-transient form in memory 22 in order to cause the relay device to perform its functions. The processor can communicate with transceivers 21, 23. The wireless transceiver 21 is configured to communicate with wireless transceiver 17 of the sensing device. The uplink transceiver may use any suitable wired or wireless protocol for establishing a link 30 with the data centre 3. For example, the uplink transceiver could be an IEEE 802.11 wireless transceiver or a wired Ethernet transceiver. The link 30 could conveniently operate over a publically accessible communications network such as the internet. The relay device, could be a cellular telephone, for example a smartphone.

The data centre 3 comprises a data store 31 and a processing station 32 which includes a processor 33 and a memory 34. The data store is connected to receive and store data received from the relay device. The processor 33 is configured to execute software stored in a non-transient form in memory 34 in order to analyse the data stored in the data store 31 and to implement predetermined actions when certain conditions are met in the data, for example by issuing an alert to an external consumer, as indicated at 40.

FIGS. 2 and 3 show examples of how the sensor device 1 can be configured and attached to a subject. The subject's skin is shown at 50. The sensor device 1 in each figure comprises four sensors 10, 11, 12, 13. Two sensors 12, 13 are positioned adjacent to an inner major surface of the sensor device. The inner major surface is directed towards the subject's skin. Two sensors 10, 11 are positioned adjacent to an outer major surface of the sensor device. The outer major surface is directed away from the subject's skin and towards the environment around the skin. Sensors 10 and 12 are configured to sense the same parameter as each other, e.g. temperature. Sensors 11 and 13 are configured to sense the same parameter as each other, e.g. humidity. It has been found that by having one sensor for a parameter exposed essentially to the environment and one nearby sensor for the same parameter exposed essentially to the skin of the subject the measurement of the parameter in respect of the subject's skin can be calibrated against, or adjusted for, the atmospheric value for the same parameter. This can result in a measure of the parameter in respect of the subject's skin that is more representative of the subject's physiology than a single measure of the parameter on the skin surface.

To get a good measure of the parameter on the skin surface it is desirable for the sensing device to be held against the skin. This may be achieved by a layer of adhesive 51 (see FIG. 2) bonding the sensing device to the skin or by a strap 52 (see FIG. 3) which is attached to the sensing device and embraces part of the subject's body so as to hold the sensing device in contact with the surface of the subject's skin.

In operation, the sensing device is fixed against the skin of a subject, e.g. with adhesive or a strap. The processor 15 of the sensing device executes the software code stored in memory 16. This causes it to, from time to time, gather measurements from the sensors 10-13 and an accelerometer 18 (see FIG. 1). The processor may process the measurements locally to the sensing device, for example by averaging or smoothing the measurements or by discarding spurious or redundant measurements. The processor may store the measurements temporarily in memory 16, e.g. until a link is established with relay device 2 or until a predetermined number of measurements have been gathered or until a predetermined time has elapsed since the last transmission of measurements to the relay device. From time to time the sensor device transmits the measurements to the relay device 2 by means of transceiver 17.

The processor 20 of the relay device causes the transceiver 23 to transmit the measurements to the data centre 3. The processor 20 may store the measurements temporarily in memory 22, e.g. until a link is established with the data centre 3 or until a predetermined number of measurements have been gathered at the relay device or until a predetermined time has elapsed since the last transmission of measurements from the relay device to the data centre. The processor 20 may also process the measurements locally, for example to compare them to predetermined forms whose characteristics are stored in memory 22, and if such forms are detected it may present an alert to a user by means of a user interface device such as a display 24 or a loudspeaker.

At the data centre, received measurements are stored in database 31.

This architecture can be used to monitor the physiological state of a subject who is suffering from a disease. It may also be used to monitor a subject who is not showing symptoms of a disease in order to provide an alert if that subject develops symptoms that may be indicative of the disease. These applications are particularly relevant to the treatment and detection of infectious diseases. The use of the architecture of FIG. 1 for these functions will be described in more detail below.

The sensing device can be equipped with sensors 10-13 for any parameters relevant to the condition that is to be studied. Examples include temperature sensors (e.g. thermistors or thermocouples), humidity sensors (e.g. resistive or capacitive humidity sensors), light sensors (e.g. photodiodes) and conductivity sensors (e.g. by means of resistance measurement) for estimating galvanic skin response and/or sensors for respiration rate, heart rate, perspiration state, blood pressure, blood oxygen content and hydration level. In each case, there may be a first sensor for the relevant parameter that is adjacent to the inner surface of the device and a second sensor for the same parameter that is adjacent to the outer surface of the device. The sensing device can also include an accelerometer 18, for example a piezoelectric or gyroscopic accelerometer. The accelerometer may be a single axis accelerometer, e.g. operating in an axis transverse to the inner major face of the device, or a multi-axis accelerometer. Examples of the quantities that can be estimated by means of the device are as follows:
The real-time temperature of the subject's skin, either as an absolute value or adjusted by means of a predetermined algorithm to compensate for environmental temperature. For example, the representative skin temperature may be estimated as Tₛ+a (b−Tₑ) where Tₛ is the temperature measured by a skin-facing sensor, Tₑ is the environmental temperature measured by a sensor on the outer major surface of the device and a and b are constants.
Estimated heat flow from the patient, obtained by comparing Tₛ and Tₑ.
The activity state of the subject as indicated by data received from the accelerometer. For example the accelerometer may indicate substantial variation in acceleration over a period of, e.g., 5 seconds, which may be indicative of the subject being active; or the accelerometer may indicate no substantial variation in acceleration over a period of, e.g., 5 minutes, which may be indicative of the subject being asleep. The processor 15 may store a plurality of threshold acceleration values, each having an associated time period, number and state, and may estimate the user to be in the most active of those states for which the measured acceleration values have exceeded the respective threshold for at least the respective number of times during the immediately preceding respective time period.
The temperature of the subject taken at a time when the accelerometer indicates the subject to be in a particular state. For example, it has been found that a particularly representative measurement of the subject's body temperature can be taken when the user is asleep. The processor may estimate that the subject is asleep by means of data from the accelerometer (e.g. as described in the preceding paragraph) and may then measure the subject's temperature, either as an absolute value or adjusted by means of a predetermined algorithm to compensate for environmental temperature
An estimate of the basal body temperature of the subject. This may be determined as the lowest absolute or adjusted temperature value taken over the course of a complete session of sleep, or by interpolating between measured values over the course of that session so as to estimate the lowest value attained by the subject. It has been found that a basal body temperature estimate can be highly significant for the monitoring or detection of disease conditions because, unlike instantaneous body temperatures, it can be measured with a high degree of accuracy in the manner described above.
Humidity at the subject's skin, either as an absolute value or adjusted by means of a predetermined algorithm to compensate for environmental temperature. This may be used to estimate whether the subject is dehydrated.
An estimate of the quality of the subject's sleep over a period of time, e.g. a sleep session. This may be estimated from the amount of motion detected by the accelerometer during the sleep session, e.g. the proportion of the time windows of a predetermined duration extending over the sleep session during which no acceleration greater than a predetermined value is detected.

These and other quantities can then be used to assist in monitoring the progression of a disease from which the subject is suffering, or to help detect the onset of symptoms of a disease in a patient who is being pre-emptively monitored. One additional source of information that may be used for estimating the presence of disease symptoms is subjective information input by the patient indicating the subject's own perception of their physiological state. This may be provided by a user interface device on the sensor device 1 or on the relay device 2. For example, the relay device may display a message asking a subject to indicate how they feel on a scale from 1 to 10. The user may input a response and that input may form an input to the algorithm that estimates the likelihood of disease.

The measurements are transmitted from the sensor device via the relay device to the data centre. At the data centre the measurements are analysed from time to time, for instance every time measurements are received, or every 2 to 6 hours. The analysis proceeds by comparing the measurements against a set of criteria the definitions of which have been previously stored in memory 34. Examples of the analysis are as follows:
The measurements can be analysed to determine whether a measured parameter, or a value derived by calculation from one or more measured parameters, exceeds a threshold. For example, the analysis could determine whether the measured skin temperature of the subject exceeds a threshold. The threshold could be an absolute value pre-stored in the memory 34 or could be determined by the data centre based on previous measurements. For example, the temperature threshold could be the average of the maximum temperatures measured for the same subject over each of the previous three days. In that way the system can identify abnormalities related to a particular individual rather than to the population in general.
The measurements can be analysed to determine whether a time series of values of one or more measured parameters, or values derived by calculation from one or more measured parameters, indicates a variation that matches a predetermined form. For example, FIG. 4 illustrates possible measurements of skin temperature, adjusted for environmental temperature, that might be taken from a number of subjects over a period of three days. The temperature for a subject A follows a normal variation. The temperatures for subjects B and C increase above a threshold 60 which has previously been determined to be indicative of a disease condition. On one metric the fact that the temperatures for subjects B and C exceed the threshold may be sufficient to trigger an alert. However, it will be seen that the pattern of temperature increase in the subjects is different. The region bounded at 61 indicates a pattern of increase that has previously been determined, from measurements in a significant population of subjects, to be characteristic of the onset of a specific disease. Since the temperature rise for subject B matches that profile subject B may be suspected to be suffering from that disease, and directed to undergo further tests with a view to establishing whether that is the case. This mechanism might be used to distinguish a temperature rise due to a less significant disease such as a cold or influenza from a temperature rise due to a more significant disease such as Ebola or malaria.
Multiple instantaneous or time-series measures may be assessed by means of a predetermined algorithm which generates for each measure a score that indicates the extent to which that measure is indicative of disease. Those scores may then be combined to provide an overall score which indicates an estimate of the subject's wellness. For example, the measurements may be analysed to identify whether there is evidence of a deterioration in a subject's quality of sleep (e.g. indicated by increased movement detected by the accelerometer 18 during a sleep session). A measure of quality of sleep (e.g. a time-averaged, thresholded acceleration average during a sleep period) may be compared with the same measure as determined over previous periods. A deterioration in that measure may be taken as an indicator of increased likelihood of disease. Similarly, an estimate of skin humidity may be assessed to provide an additional indicator of potential infection.
A user may wear multiple sensor devices like sensor device 1, each on a different part of the body. For example, the devices may be located at any of the wrist, upper arm, chest, armpit, neck, ankle and ear. The measurements from all those devices can be sent back to the data centre. Time-series of measurements from each device can be compared to identify data indicative of a disease, and the relative pattern of variation between the measurements from different sensor devices might provide a suggestion that the subject is suffering from one disease rather than another. Data can be gathered from a significant population of subjects to identify characteristics of the onset of a specific diseases: for example that a substantial rise in temperature at the ear precedes a rise in temperature at the wrist. If such a characteristic is detected in the data from a subject then that may be taken as suggesting that the user might be suffering from the corresponding disease rather than another disease. A further advantage of the subject wearing multiple sensors is that if one sensor is dropped or lost continuity of measurement can be maintained. This can be of particular significance for prompt detection of the onset of a disease condition. Sensors in particular locations on the body may provide better measurement accuracy for certain physical parameters. For example, measurement of temperature at the armpit and also at the wrist may give additional insight into blood flow compared to a temperature measurement at either site alone. In another example, synchronous measurement of pulse at the armpit and wrist may give insight into pulse transit time, which correlates well with blood pressure. (See http://thorax.bmj.com/content/54/5/452.full).

Some ways in which the system of FIG. 1 can be applied will now be described.

A patient suffering from a communicable disease may be isolated in a unit such as a barrier tent, with minimal direct contact to the exterior of the tent. The patient can wear the sensor device 1. The sensor device is self-contained and can take measurements without needing to receive power from outside the tent. The measurements taken by the device can be transmitted wirelessly through the wall of the tent to the relay device 2. In that way the measurements can be taken proximally to the patient but without the need to penetrate the wall of the tent. That can increase the accuracy of the measurements without compromising the isolation of the patient. The measurements can be reviewed and analysed on the relay device or at the data centre.

A healthcare worker who has treated a patient may wear the sensor device 1. Measurements can be taken from the sensor device and fed back to the data centre. The data centre can generate an alert, for example by sending a message to the relay unit or to a control centre, if a predetermined condition is detected in the worker. This approach has a number of advantages over conventional ways of monitoring workers.
Because the sensor device is worn continually by the worker the data centre can build up data indicating the typical physiological values obtained for the worker when he or she is well. This permits the data centre to treat deviations of relatively low magnitude from the normal values as significant, and hence to provide an earlier warning of the possible onset of disease than with other methods.
If the worker loses the sensor device or takes it off then the values received from the device will change and will be unrepresentative of the data typically received when the device is being worn. For example the received skin temperature values might be unrepresentative of typical absolute human skin temperature, or might not follow the normal diurnal variation of human skin temperatures, or the temperature or humidity readings from both sides of the sensor device might vary by less than would be expected when the device is being worn. This permits the data centre to identify that the worker is not wearing the device. In response to that detection the data centre can generate an alert. This can help to ensure that the worker is complying or adhering to the monitoring regime.
The data centre can automatically raise an alert if measurements suggestive of a disease condition are identified. This avoids the possibility that the worker might not report themselves.
Workers who are treating patients in isolation frequently wear barrier suits. The environment inside a barrier suit can become hot and humid, which can make it difficult to obtain accurate measurements of the worker's own temperature. By using a temperature sensor of the type shown in FIGS. 1 to 3, in which the sensing device has a first sensor close to the skin and a second sensor exposed to the environment, the skin temperature measurement can be adjusted in dependence on the environmental temperature measurement. This allows a more representative value to be obtained for the subject's skin temperature or humidity irrespective of the environmental conditions in the worker's barrier suit. The entire sensor device could be incorporated into the barrier suit, or the barrier suit could comprise a sensor for measuring the environmental temperature or humidity within the suit and the worker could wear a sensor for sensing their skin temperature. The data from those sensors could be combined in the way described previously.
If a worker were to be diagnosed with an infection that required them to be isolated then they could continue wearing the same sensor in isolation. That can help to reduce the need to equip the worker when the go into isolation, reducing the prospect of other workers being exposed to infection.
There could be multiple workers in a healthcare team who are wearing sensors of the type described above. Those workers' sensors could pass measured data to a common relay device 2, which then forwards that data to the data centre for analysis. This reduces the number of relay devices that are required.
If the isolation of a patient suffering from an infectious disease were to be breached then multiple healthcare workers could be infected at a similar time. A variation of relatively small magnitude in a parameter as measured for a number of related healthcare workers could be treated by the data centre as statistically significant and could cause the data centre to generate an alert of potential infection. This can permit infection to be detected earlier than would otherwise be the case, assisting in outbreak control. In addition to healthcare workers, this system may be of use to groups of border workers, public transport workers and the like, who may be exposed to an infectious individual at a similar time.

In the examples described above the main data analysis is performed at the data centre 3, but it could be performed at the relay unit 2 or at the sensor device 1.

The system described above may be used for detecting that sensed data is indicative of a disease condition. That disease condition may be the onset of initial symptoms of a disease, or an exacerbation of an ongoing or chronic disease. For example, in the case of chronic obstructive pulmonary disease (COPD) during an exacerbation symptoms typically worsen for three to five days before treatment is required. By detecting worsening symptoms during that period it may be possible to intervene with therapies that avoid the need for the patient to undergo a stay in hospital.

Some non-limiting examples of diseases that could be indicated, diagnosed or monitored for using the system include chronic obstructive pulmonary disease (COPD), cystic fibrosis (CF), diabetes, hypoglycaemia, sleep disturbance, sleep apnoea, chronic pain, infection (e.g. by bacterial, viral, prion, protozoal, fungal or parasitic agents), sepsis, polycystic ovary syndrome (PCOS), menopause, asthma, insomnia, schizophrenia, coronary heart disease, narcolepsy, restless legs syndrome, rheumatoid arthritis, inflammatory bowel disease (IBD), lupus, periodic fever syndromes and cancers such as lymphoma, leukaemia and renal cancer. The sensor and the carrier may be applied to humans or animals.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A physiological monitoring system comprising:
a plurality of first sensor devices, each for attachment to the skin of a respective subject, each sensor device comprising a first body, a first temperature sensor and a second temperature sensor at, wherein the first temperature sensor is located adjacent to a first surface of the first body and the second temperature sensor is located adjacent to an opposing surface of the first body;
and
a processing device configured for analysing data sensed by the first and second temperature sensors by means of a predetermined algorithm to compensate for environmental temperature and to estimate whether the data is indicative of a disease condition, the analysis being such as to generate an alert if the data received from multiple ones of the sensor devices demonstrate a contemporaneous trend indicative of a disease condition.

2. (canceled)

3. A physiological monitoring system as claimed in claim 1, wherein each sensor device further comprises an accelerometer.

4. A physiological monitoring system as claimed in claim 2, wherein the system is configured to selectively analyse data gathered by the first and second temperature sensors at times dependent on data gathered by the accelerometer.

5. A physiological monitoring system as claimed in claim 3, wherein the system is configured to, in dependence on data sensed by the accelerometer during a sleep session of each subject, form an estimate of the quality of the subject's sleep during that session and to estimate whether the data is indicative of a disease condition in dependence on that sleep quality estimate.

6. (canceled)

7. A physiological monitoring system as claimed in claim 3, wherein the system is configured to selectively analyse temperature data gathered at times of relatively low acceleration as sensed by the accelerometer.

8. A physiological monitoring system as claimed in claim 5, wherein the system is configured to form an estimate of the lowest temperature attained by a wearer of the device during a sleep session and the estimation of whether the data is indicative of a disease condition is dependent on that temperature estimate.

9. A physiological monitoring system as claimed in claim 5, wherein the system is configured to form an estimate of the basal body temperature of a wearer of the device during a sleep session and the estimation of whether the data is indicative of a disease condition is dependent on that temperature estimate.

10. A physiological monitoring system as claimed in claim 1, wherein the system is configured to store data previously sensed by each sensor device and the estimation of whether the data is indicative of a disease condition is dependent on detecting a deviation of a predetermined form between data currently sensed by each sensor device and that previously sensed data.

11. A physiological monitoring system as claimed in claim 8, wherein the deviation of a predetermined form is a deviation by greater than a threshold from an average formed over at least part of the previously sensed data.

12. A physiological monitoring system as claimed in claim 1, wherein the system is configured to store data previously sensed in respect of multiple subjects and determined to be indicative of the onset of a disease condition and the estimation of whether the data is indicative of a disease condition is dependent on detecting a commonality of a predetermined form between data currently sensed by each sensor device and that previously sensed data.

13. A physiological monitoring system as claimed in claim 10, wherein the system is configured to generate an alert indicating the disease condition with the data for which a commonality has been detected in data currently sensed by the sensor.

14. (canceled)

15. (canceled)

16. A physiological monitoring system as claimed in claim 1, wherein the system is configured to analyse data received from the first and second temperature sensors to compare the received data with one or more data patterns whose definitions are stored as being indicative of a device that is being worn by a human subject, and to generate an alert on detecting a deviation of a predetermined form between data currently sensed by the sensor device and one or more of those patterns.

17. A physiological monitoring system as claimed in claim 1, wherein the system comprises a plurality of second sensor devices, each for attachment to the skin of a respective subject, each second sensor device comprising a second body, a third temperature sensor and a fourth temperature sensor, wherein the third temperature sensor is located adjacent to a first surface of the second body and the fourth temperature sensor is located adjacent to an opposing surface of the second body; the first and second sensor devices are adapted to be attached to the body of the respective subject at different locations; the system is configured to analyse data received from the first and second sensor devices in dependence on their locations on the body of the respective subject to estimate whether the data is indicative of a disease condition.

18. A physiological monitoring system as claimed in claim 1, wherein the disease condition is the onset of initial symptoms of a disease.

19. A physiological monitoring system as claimed in claim 18, wherein the disease is a viral infection.

20. A physiological monitoring system as claimed in claim 1, wherein the disease condition is an exacerbation of a chronic disease.

21. A method for monitoring a plurality of subjects, comprising:
providing each subject with a sensor device for attachment to the skin of the subject, the sensor device comprising a body, a first temperature sensor and a second temperature sensor, wherein the first temperature sensor is located adjacent to a first surface of the body and the second temperature sensor is located adjacent to an opposing surface of the body; and
analysing data sensed by the first and second temperature sensors by means of a predetermined algorithm to compensate for environmental temperature and to estimate whether the data from each sensor device is indicative of a disease condition, the analysis being such as to generate an alert if the data received from multiple ones of the sensors devices demonstrate a contemporaneous trend indicative of a disease condition.

22. (canceled)
